(19) United States
(12) Patent Application Publication
(10) Pub. No.: US 2012/0041334 A1
(43) Pub. Date: Feb. 16, 2012

(54) DEVICE FOR MEASURING THE BLADDER PRESSURE

(76) Inventors: Oliver Goedje, Strasslach (DE); Manu Malbrain, Lovenjoel (BE)

(21) Appl. No.: 13/263,813

(22) PCT Filed: Mar. 25, 2010
(86) PCT No.: PCT/EP2010/053876

§ 371 Date: Oct. 10, 2011

(30) Foreign Application Priority Data
Apr. 9, 2009 (DE) 10 2009 017 034.0

(51) Int. Cl. A61B 5/00; A61M 29/00

(52) U.S. Cl. 600/561; 604/99.04

(57) ABSTRACT

A device for measuring bladder pressure and/or for measuring intra-abdominal pressure within the body of a living being comprises a pressure measuring unit including a pressure sensor, and a urethral catheter comprising a balloon lumen, which connects a distal balloon for measuring internal pressure thereof to the pressure measuring unit, and a bladder lumen having a distal opening. The bladder lumen is, without detaching connectors, alternately connectable to a proximal urine outlet and separable therefrom, and the pressure sensor of the pressure measuring unit is connected to or is alternately connectable, without detaching connectors, to the bladder lumen.

The invention relates to a device for measuring bladder pressure and/or for measuring intra-abdominal pressure inside the body of a living being, comprising a pressure measuring unit and a urethral catheter which comprises a balloon lumen, which connects a distal balloon for measuring the internal pressure thereof with the pressure measuring unit, and a bladder lumen having a distal opening.

Various devices for measuring bladder pressure are already known from the prior art. Patent specification U.S. Pat. No. 4,538,621 thus discloses a direct measurement of bladder pressure via a liquid column.

Patent specification U.S. Pat. No. 6,447,462 B1 describes a device of the type initially mentioned. A balloon catheter with an external pressure measuring unit which is connectable thereto makes it possible to measure bladder pressure via the balloon's internal pressure. The gas pressure is transferred from the inside of the balloon to the pressure sensor via a balloon lumen. A zero adjustment to ambient pressure is possible via a vent opening. A separate lumen with a distal opening serves as a filling lumen. The measurement of bladder pressure via a balloon catheter is also described in U.S. Pat. No. 7,004,899 B1, more specifically by means of a single-lumen specific measuring catheter.

The measuring devices known from the prior art for measuring bladder pressure have the drawback that they are generally difficult to calibrate and undetected errors of measurement may thus occur. Specific measuring catheters are further generally used in conjunction with the known measuring devices. If a patient has been fitted with a conventional Foley catheter in order to ensure the discharge of urine from the bladder, it is necessary, in connection with known devices, to exchange the catheter for measuring bladder pressure.

The object of the invention is therefore to provide a device for measuring bladder pressure and/or for measuring intra-abdominal pressure within the body of a living being, which device, where possible, does not require a catheter change to determine bladder pressure, is easy to implement and use, and in particular allows simple calibration of the pressure sensor.

The object is achieved with the device according to claim 1. Advantageous embodiments are described in independent claims 2 to 11.

According to one aspect, the invention therefore relates to a device of the type initially mentioned, wherein the bladder lumen is alternately connectable, without detaching connectors, to a proximal urine outlet and, in order to measure the pressure in the bladder lumen, to a pressure sensor of the pressure measuring unit. It is thus made possible to calibrate the continuous or semi-continuous determination of bladder pressure and/or intra-abdominal pressure provided by the measurement of the gas pressure in the balloon/balloon lumen system using a preferably intermittently repeated measurement of internal bladder pressure via the liquid column in the bladder lumen.

Conventional Foley catheters used to remove urine from the urinary bladder via the urethra or via the abdominal wall can thus advantageously be used to measure bladder pressure and are to be connected merely to a suitable pressure measuring unit, preferably designed as a single-use pressure measuring unit. The pressure measuring unit is preferably connected to the urethral catheter without the use of tools by means of a quick-fixing device, for example snap fastener or a Luer lock connector for bladder lumen and balloon lumen. Such a pressure measuring unit according to the invention is designed to measure both the gas pressure in the balloon/balloon lumen system and the liquid pressure in the bladder lumen, which normally serves as a urine discharge lumen. As a result of the feature provided according to the present invention to alternately connect the bladder lumen, without detaching connectors, to a proximal urine outlet and to a pressure sensor of the pressure measuring unit, both the function of urine discharge and the function of the (semi-)continuous bladder pressure measurement including calibration can be performed by the same (per se conventional) catheter.

As an alternative, the pressure sensor may also be permanently connected to the bladder lumen, but in this case only provides suitable calibration values if the proximal urine outlet is closed; in addition such a solution requires a second pressure sensor for measuring the gas pressure in the balloon/balloon lumen system, whereas with alternate separation of the pressure sensor from the bladder lumen a single pressure sensor may be sufficient for the semi-continuous determination of bladder pressure and the intermittent calibration measurement.

Manually controllable valves and valves controlled via a control means may be provided for alternate connection of the bladder lumen to the urine outlet or the pressure sensor. In principle, a device according to the invention may already be implemented using a single three-way cock for alternate connection of the bladder lumen to the pressure sensor or the urine outlet. The necessary valves are preferably integrated in the pressure measuring unit so that all necessary sensors and valves can be connected in one step to the urethral catheter in a simple manner and without risk of incorrect assembly.

According to a preferred embodiment of the invention the device comprises an external evaluation unit. The evaluation unit is designed in such a way that it can be connected to the pressure measuring unit so that the measuring signals of the pressure sensor unit can be read out and evaluated. The evaluation unit may preferably also comprise a control channel for controlling a valve means which intermittently connects the pressure sensor to the bladder volume in order to carry out a calibration measurement. If the device is equipped with a manual valve device for connecting the pressure sensor to the bladder lumen, a channel for detecting the valve switching process may advantageously be provided so that, when the valve is switched over, the evaluation unit automatically switches over to a calibration mode or back into a continuous measurement mode.

A zero adjustment line may also be provided for carrying out a zero adjustment of the pressure sensor measuring the gas pressure in the balloon/balloon lumen system in relation to the surrounding atmospheric pressure or another reference pressure.

According to a further advantageous embodiment of the invention the device comprises a position sensor. A correction function can hereby be implemented in the evaluation unit which takes into account possible changes of the height difference between the pressure sensor and the urinary bladder during the measurement, since the hydrostatic pressure of the liquid column in the bladder lumen is included in the measurement.

According to an advantageous enhancement of the invention the device comprises a flowmeter for measuring the urine flow through the bladder lumen.

In principle, any variant of the invention described or implied within the scope of the present application may be particularly advantageous depending on the economic and technical conditions in each individual case. Unless otherwise indicated, and as far as generally technically possible, individual features of the described embodiments may be exchanged or combined with one another as well as with features known per se from the prior art.

The invention will be explained herein-below by way of example with reference to drawings. Several preferred embodiments will be described, although the invention is not limited thereto.

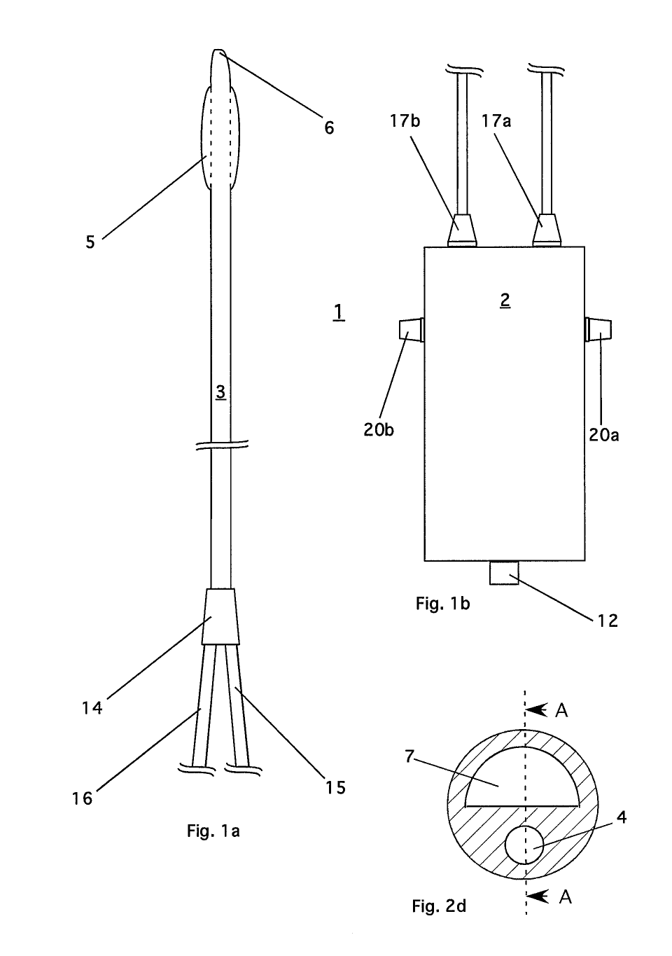

FIGS. 1a-b schematically show a device according to the invention for measuring bladder and/or intra-abdominal pressure.

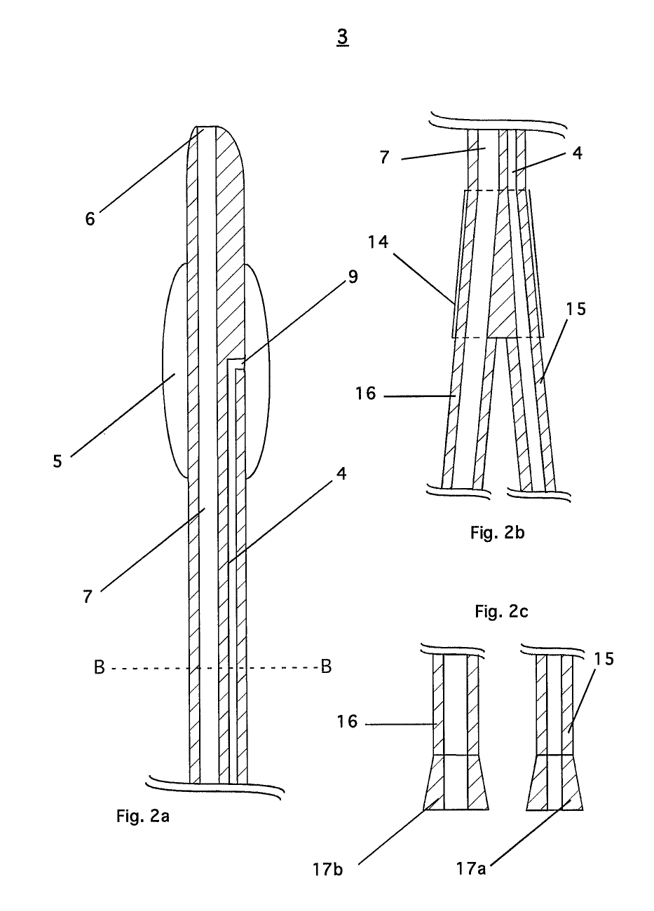

FIGS. 2a-d are schematic sectional views of the urethral catheter of a device according to the invention.

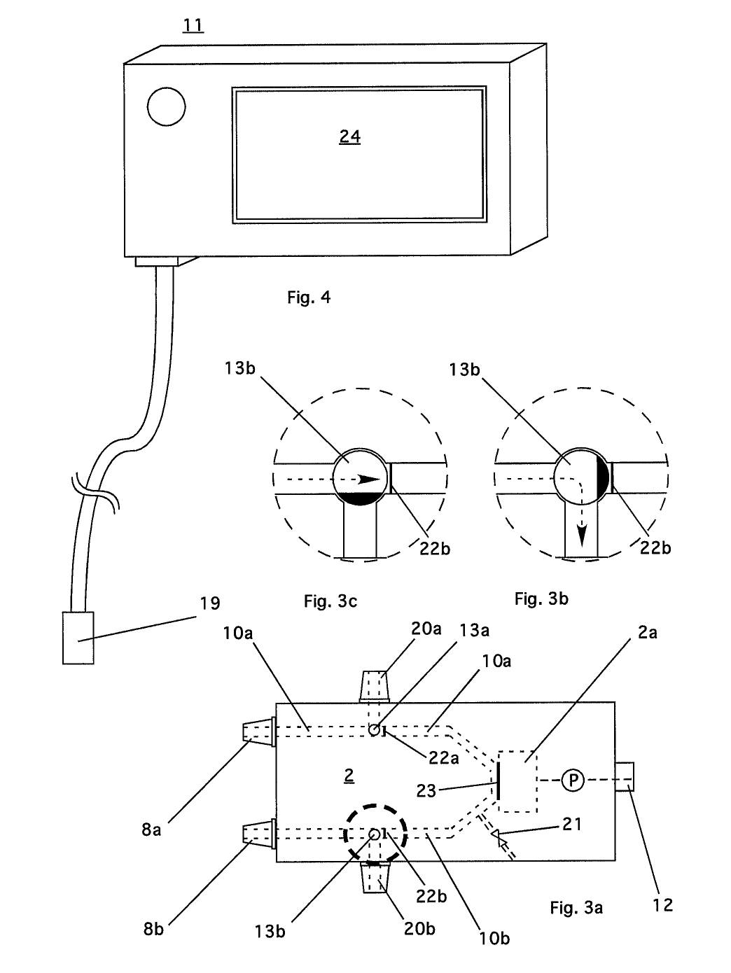

FIG. 3a schematically shows the separate pressure measuring unit, which is connectable to a Foley catheter, of a device according to the invention.

FIGS. 3b-c are schematic detailed views of the position of a three-way cock of the device according to the invention.

FIG. 4 is a schematic view of the evaluation unit of the device according to the invention.

The device 1 illustrated in FIGS. 1a and 1b consists of the urethral catheter 3 illustrated in FIGS. 2a-d and the separately connectable pressure measuring unit 2 illustrated in FIG. 3a. The urethral catheter 3 also comprises, in addition to the balloon lumen 4 connected to the distal balloon 5, a urine discharge lumen (bladder lumen) 7 connected to a distal opening 6. The channel separation 14 branches the urethral catheter 3 into a pressure conduit 15 as a continuation of the balloon lumen and a drainage conduit 16 as a continuation of the urine discharge lumen 7. The pressure conduit 15 and the drainage conduit 16 each comprise, at their ends opposite the channel separation 14, a coupling piece 17a and 17b, respectively, which may be designed as Luer lock connectors.

The pressure measuring unit 2 comprises a housing with an inner pressure sensor unit 2a, two lumen connections 8a, 8b which are each formed as a counter piece to the coupling pieces 17a and 17b, respectively, and two three-way valve devices 13a, 13b for feeding and carrying off the pressure measuring gas and for removing the urine.

FIGS. 2a to 2d show sectional views of the urethral catheter 3 with a balloon lumen 4 and a urine discharge lumen 7. FIG. 2a is an interrupted longitudinal section (sectional plane indicated in FIG. 2d by dashed line A-A). FIGS. 2b and 2c are continuations of the longitudinal section from FIG. 2a, on either side of the break lines, respectively. FIG. 2b shows a cross-section (sectional plane indicated in FIG. 2a by dashed line B-B). The gas volume required to fill the balloon 5 is conveyed via the pressure conduit 15 through the balloon lumen 4 to the distal balloon 5. The balloon 5 may further serve as a blocker balloon for fixing the catheter 3 in the patient. A possible variant with an additional blocker balloon comprising a separate, further balloon lumen which is arranged proximally from the balloon 5 serving purely as a measurement balloon is not illustrated, but also lies within the scope of the present invention. The gas volume required to fill the balloon is supplied via the gas connector 20a.

Urine to be removed enters at the distal opening 6 and passes via the urine discharge lumen 7 towards the discharge conduit 16 and beyond.

The pressure measuring unit 2 illustrated in FIG. 3 comprises two lumen connectors 8a and 8b. The lumen connector 8b connectable to the coupling piece 17b is alternately connectable by means of the three-way valve 13b to the urine discharge 20b (valve position from FIG. 3b) to discharge the urine, and to the pressure line 10b (valve position from FIG. 3c) to take a calibration measurement. The pressure line 10b is part of a closed system which is filled with a liquid or gaseous transfer medium. The transfer medium transfers the pressure to the measurement diaphragm 23 of the pressure sensor unit 2a. A gaseous transfer medium affords the advantage that it can be zeroed in relation to atmospheric pressure in a simple manner via a vent 21. The pressure line 10b is closed to the urine by a diaphragm 22b. The three-way valve device 13b is preferably controlled electronically via a control channel of the external evaluation unit 11.

The lumen connector 8a connectable to the coupling piece 17a is alternately connectable by means of the three-way valve device 13a to the gas connection 20a to fill/evacuate the balloon 5 and to the pressure line 10a to take a (semi-)continuous measurement of the internal balloon pressure. The pressure line 10a is also part of the aforementioned closed system filled with transfer medium. The pressure line 10a is closed to the balloon filling gas by a diaphragm 22a. The three-way valve device 13a is also preferably switched electronically via a control channel of the external evaluation unit 11.

The measurement signals of the pressure sensor unit 2a are output via a pressure measurement channel P to the evaluation unit 11, which is adapted to calibrate a calibration of the internal balloon pressure measured via the pressure line 10a by comparison with the urine pressure measured intermittently via the pressure measurement line 10b. As mentioned, it is advantageous if the three-way valve devices 13a, 13b can be controlled for this purpose by the evaluation unit 11. The measurement and control channels may be guided via a cable which is connectable by means of a plug connector 12, 19. A wireless connection is also possible. The measured pressure is displayed via the display 24, which may also serve as a graphic user control and, for this purpose, can also be designed as a touchscreen, for example.

1. A device for measuring bladder pressure and/or for measuring intra-abdominal pressure within the body of a living being, comprising:
a pressure measuring unit including a pressure sensor; and
a urethral catheter comprising a balloon lumen, which connects a distal balloon for measuring internal pressure thereof to the pressure measuring unit, and a bladder lumen having a distal opening (6)
wherein the bladder lumen is, without detaching connectors, alternately connectable to a proximal urine outlet and separable therefrom, and the pressure sensor of the pressure measuring unit is connected to or is alternately connectable, without detaching connectors, to the bladder lumen.

2. The device according to claim 1, wherein the pressure sensor is alternately connectable to the balloon lumen and to the bladder lumen.

3. The device according to claim 1, wherein the pressure measuring unit is designed as a separate single-use pressure measuring unit which is connectable to the bladder catheter.

4. The device according to claim 1, wherein the pressure measuring unit is connectable to the urethral catheter, without the use of tools, by means of a quick-fixing device.

5. The device according to claim 1, wherein the pressure measuring unit further comprises a zero adjustment line for zero adjustment in relation to the ambient pressure or another external calibration pressure.

6. The device according to claim 1, further comprising an external evaluation unit which is connectable to the pressure measuring unit.

7. The device according to claim 6, wherein the external evaluation unit is set up in such a way that the pressure measuring unit can be calibrated for the pressure measurement in the balloon lumen of the urethral catheter via the liquid column of the bladder lumen.

8. The device according to claim 6, further comprising a three-way valve device which is controllable by the external evaluation unit in such a way that, for calibration, the bladder lumen can be sealed from the urine outlet and is simultaneously connectable to the pressure sensor.

9. The device according to claim 6, wherein the external evaluation unit is adapted to repeat the calibration intermittently for pressure measurement in the balloon lumen.

10. The device according to claim 1, wherein the device comprises a position sensor by which the position of the pressure measuring unit relative to the patient can be determined.

11. The device according to claim 1, which further comprises a flowmeter for measuring the urine flow through the bladder lumen.
